(19) United States
(12) Patent Application Publication
(10) Pub. No.: US 2022/0088323 A1
(43) Pub. Date: Mar. 24, 2022

(54) FLUID PRESSURE INDICATORS FOR MEDICAL DEVICES

(71) Applicant: Boston Scientific Scimed, Inc., Maple Grove, MN (US)

(72) Inventors: Ryan EVERS, Billerica, MA (US); Andrew PIC, Northboro, MA (US); Collin MURRAY, Bolton, MA (US); Matthew R. JAGELSKI, Milford, MA (US); Daniel CONGDON, Hudson, MA (US)

(73) Assignee: Boston Scientific Scimed, Inc., Maple Grove, MN (US)

(21) Appl. No.: 17/479,281

(22) Filed: Sep. 20, 2021

Related U.S. Application Data
(60) Provisional application No. 63/081,078, filed on Sep. 21, 2020.

(51) Int. Cl. A61M 13/00; B65D 83/20; B65D 83/22

(52) U.S. Cl. CPC A61M 13/00; B65D 83/207; A61M 2205/8225; A61M 2205/14; A61M 2205/583; B65D 83/228

(57) ABSTRACT

A device for delivering pressurized fluid includes a handle having a lumen configured to receive a container of pressurized fluid, a cap to close the lumen and an indicator to move from a first position to a second position. The first position indicates disconnection of the container from a fluid path, and the second position indicates connection of the container to the fluid path.

### CROSS-REFERENCE TO RELATED APPLICATIONS

This application claims the benefit of priority of U.S. Provisional Patent Application No. 63/081,078, filed Sep. 21, 2020, the entirety of which is incorporated herein by reference.

### TECHNICAL FIELD

The disclosure relates generally to medical systems and devices for delivering pressurized fluids, and in examples, to methods and tools for indicating connection of a pressurized fluid source to a medical device.

### BACKGROUND

Fluid delivery systems and devices are used to supply various fluids, such as a gas, during medical procedures. These procedures may include supplying fluids within a range of appropriate pressures and/or flow rates. These fluids may include hemostatic agents optimally delivered to tissue at an appropriate pressure and/or flow rate, for the particular application.

Handheld medical fluid delivery systems often require delivering a fluid from a high pressure storage tank, such as a cartridge or similar housing. Such cartridges may be loaded into a handle of the delivery system and may energize or charge the delivery system when a seal on the cartridge is pierced by a pierce pin or similar device on the delivery system. These fluids may be under high pressures and may cause injury if the medical system is not used properly. In some instances, a user is unaware when the medical system is charged with the pressurized fluid. In other instances, a user may be unaware if the cartridge is properly attached to the medical system and/or if fluid from the cartridge is being supplied to the medical system. The disclosure may solve one or more of these problems or other problems in the art. The scope of the disclosure, however, is defined by the attached claims and not the ability to solve a specific problem.

### SUMMARY OF THE DISCLOSURE

According to an aspect, a device for delivering pressurized fluid includes a handle having a lumen configured to receive a container of pressurized fluid, a cap configured to close the lumen, and an indicator configured to move from a first position to a second position, wherein the first position indicates disconnection of the container from a fluid path, and the second position indicates connection of the container to the fluid path.

The device may further include the container, and the lumen may be outside the fluid path.

The device may further include a pierce pin within the handle and configured to pierce a seal of the container.

The indicator may include one or more markings configured to indicate when the container is in the second position, wherein the one or more markings may be visible to a user when the container is in the second position and may not be visible to the user when the container is in the first position.

The indicator may be connected to the cap via an elongate member, and wherein the elongate member may be radially offset from a longitudinal axis of the handle.

The indicator includes an annular member, and wherein the indicator is configured to move relative to the handle and the container as the indicator moves between the first position and the second position.

An opening in the annular member may accommodate the container.

A diameter of the indicator may be greater than an outer diameter of the container and less than an inner diameter of the handle.

The indicator may include one or more springs configured to bias the indicator in the first position.

The pressurized fluid may be configured to overcome a spring force of the one or more springs to move the indicator from the first position to the second position.

The indicator may include a movable member configured to rotate about an axis within a housing based on a pressure of the pressurized fluid flowing into the device from the container.

The indicator may include a projection configured to move in a direction radially outward of the handle when the pressurized fluid flows through the fluid path.

The indicator may be configured to move from the second position to a third position, the third position being between the first position and the second position to operate the device.

The device may further include an actuator configured to release the fluid from a downstream end of the device, and wherein the actuator may be configured to be actuatable only when the indicator is in the second position.

The indicator may be configured to indicate the pressurized fluid is flowing from the container at a rate sufficient to operate the device when the indicator is in the second position.

According to another aspect, a device for delivering pressurized fluid includes a handle, a cap configured to close an opening in the handle, and an indicator attached to the cap, wherein the indicator includes a ring-shaped member configured to move relative to the handle when the cap is attached to the handle, and wherein the indicator is configured to indicate connection of a fluid-containing container to a fluid path of the handle.

An opening in the ring-shaped member may accommodate the container.

The cap and the indicator may be each configured to rotate relative to the handle.

An elongate member may extend from and may be attached to the cap at a first end, wherein the elongate member may be attached at a second end, opposite the first end, to the indicator, and wherein the elongate member may be offset from a central longitudinal axis of the cap.

According to another aspect, a method for delivering pressurized fluid to a device includes inserting a container of pressurized fluid into a handle of the device, moving an indicator from a first position to a second position, wherein the first position indicates disconnection of the container from a fluid path of the device, and the second position indicates connection of the container to the fluid path of the device, and actuating the device when the indicator is in the second position.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a schematic of a delivery system according to an exemplary embodiment;

FIG. 2 is a schematic of an indicator of the delivery system of FIG. 1 according to an exemplary embodiment;

FIGS. 3A, 3B, and 3C are side views of attaching a containment device to the delivery system of FIG. 1 according to an embodiment;

FIG. 4 is a cross-section of a handle of the delivery system of FIG. 1 having an indicator according to another embodiment;

FIG. 5 is a cross-section of a handle of the delivery system of FIG. 1 having another indicator according to an embodiment;

FIGS. 6A and 6B are side views of an indicator according to an embodiment;

FIGS. 7A and 7B are side views of an indicator according to another embodiment; and

FIG. 7C is a cross-sectional view of the indicator of FIGS. 7A and 7B according to an embodiment.

### DETAILED DESCRIPTION

The disclosure is described with reference to exemplary medical systems for dispensing an agent (such as a hemostatic agent or any therapeutic agent) using a pressurized fluid. The indicators associated with the medical systems may improve the functionality and/or the safety of the medical systems by informing users when the systems are pressurized with a fluid. This may reduce the potential for the unintended depressurization of the medical system during use, breakage of the containment device or the handle, or other unintended failures of the device during use. In examples, indicators provided on or in the medical system may provide the user with information relating to a proper connection between the cartridge and the medical system. However, it should be noted that reference to any particular procedure is provided only for convenience and not intended to limit the disclosure. A person of ordinary skill in the art would recognize that the concepts underlying the disclosed device and application method may be utilized in any suitable procedure, medical or otherwise. The disclosure may be understood with reference to the following description and the appended drawings, wherein like elements are referred to with the same reference numerals.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

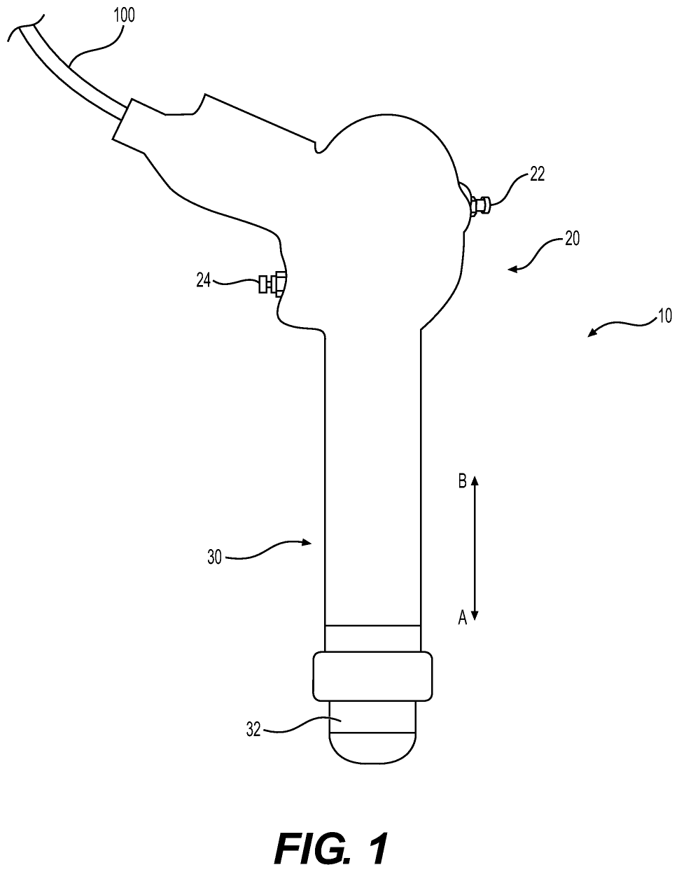

For ease of description, portions of the device and/or its components are referred to as proximal and distal portions. It should be noted that the term “proximal” is intended to refer to portions closer to a user of the device or upstream in a propellant fluid path (in the direction of arrow A in FIG. 1), and the term “distal” is used herein to refer to portions further away from the user or downstream in the propellant fluid path (in the direction of arrow B in FIG. 1). Similarly, extends “distally” indicates that a component extends in a distal direction, and extends “proximally” indicates that a component extends in a proximal direction. Further, as used herein, the terms “about,” “approximately” and “substantially” indicate a range of values within +/−10% of a stated or implied value. Additionally, terms that indicate the geometric shape of a component/surface refer only to approximate shapes.

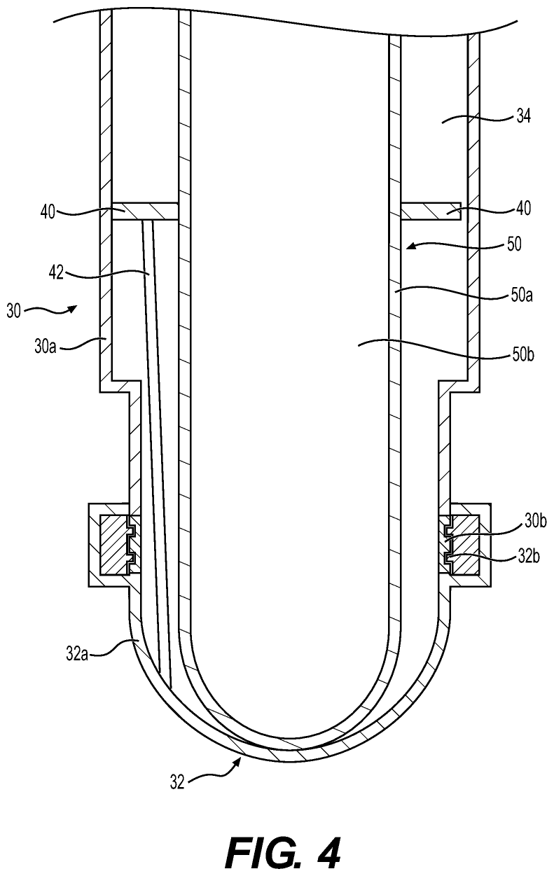

Referring to FIG. 1, a delivery system 10 according to an embodiment is shown. Delivery system 10 includes an application device 20, e.g., a hand-held device, having a handle 30 at a proximal end, and one or more triggers or actuators 22, 24 configured to actuate delivery system 10 to release a propellant fluid. A tube (e.g., a catheter) 100, or an application tip, may be attached to a distal outlet of delivery system 10 to aid in supplying the propellant fluid and/or a mixture of the propellant fluid and a hemostatic agent to a desired location. As will be described herein, a containment device 50 (e.g., a cartridge) may be contained within handle 30 (FIG. 4). A cap 32 (e.g., a cradle) may be releasably attached to handle 30 and may control and/or assist in the attachment of containment device 50 to application device 20 within handle 30. An example of delivery system 10 is in U.S. application Ser. No. 16/589,633, filed Oct. 1, 2019, which is incorporated by reference herein in its entirety.

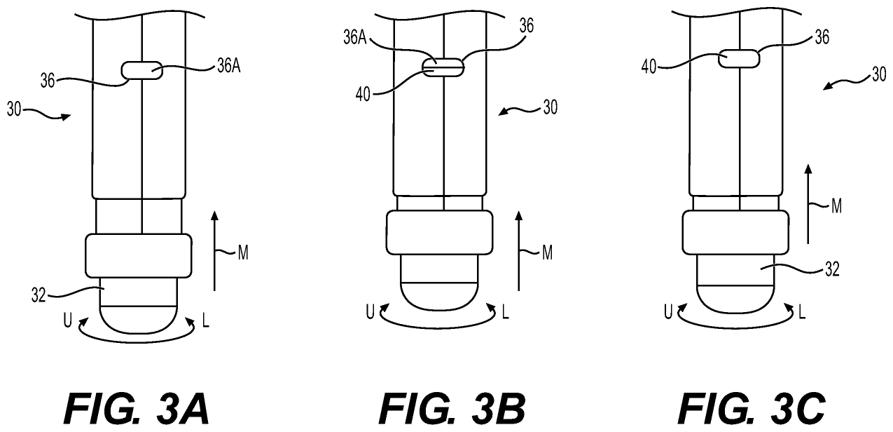

As shown in FIGS. 3A-3C, handle 30 may include a window 36 (e.g., an opening) in a sidewall thereof. Window 36 may be any shape, e.g., oval, rectangular, or the like, and may allow a user to view an inner cavity of handle 30 and/or a device within the inner cavity of handle 30, as will be described herein. Window 36 may include a covering, e.g., a clear, semi-transparent, or colored covering, to seal the inner cavity of handle 30 from an outer surface of handle 30.

With reference to FIGS. 1 and 4, containment device 50 is configured to contain a propellant fluid, such as a gas, e.g., carbon dioxide or any other propellant gas or fluid known in the art. While shown as a cylinder, containment device 50 may be any shape, such as a torpedo-shape, a sphere, or any other shape known in the art for containing gas. For example, containment device 50 could be a carbon dioxide cylinder for insertion into a lumen 34 of handle 30 (FIG. 4). Containment device 50 includes one or more outer walls 50a defining one or more inner chambers 50b, inner chamber(s) 50b configured to contain the propellant fluid. Walls 50a of containment device 50 may be formed of any material suitable for containing the fluid, such as but not limited to a metal alloy, a ceramic, or other material known in the art. The fluid contained in inner chamber 50b of containment device 50 may be under pressure. Accordingly, walls 50a are formed of a material and/or a thickness suitable to contain the fluid at a pressure of, for example, at least approximately 1000 pounds per square inch (PSI), or approximately 850 PSI. For example, gases which may be contained in containment device 20 include carbon dioxide (CO₂) having a vapor pressure of approximately 2,000-8,000 kPa at typical device temperatures, or nitrogen (N₂) having a vapor pressure less than 40 MPa at typical device temperatures. It will be understood that these gases are examples and are not limiting to the types of gases contained in containment device 50.

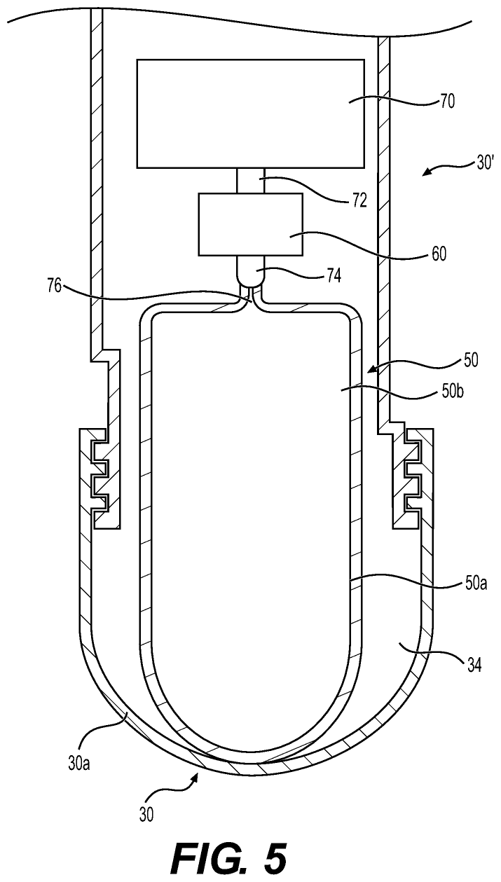

With continued reference to FIGS. 1 and 4, application device 20 is attached to containment device 50 by inserting containment device 50 into lumen 34 of handle 30. For example, an inlet (e.g., inlet 76 of a tube 74 in FIG. 5) of application device 20 may be connected directly to an output, such as a protuberance (unlabeled in FIG. 5) of containment device 50 using a threaded connection, pressure washer adapter, or the like. The protuberance of containment device 50 may extend into inlet 76 of application device 20 and connect, directly or indirectly, to a regulator (e.g., regulator 70). Directly connecting application device 20 to containment device 50 may be suitable for, e.g., a small-volume containment device 20 containing approximately 5 g to 75 g of compressed gas, or preferably approximately 12 g to 40 g of compressed gas, to allow for greater portability of delivery system 10.

Referring to FIG. 1, actuation of one or both of actuating devices 22, 24 of application device 20 causes the fluid to exit delivery system 10 to a target site via tube 100. It will be understood that only one actuating device 22, 24 may need to be actuated in some embodiments. Actuation of one or both actuating devices 22, 24 releases a buildup of pressure within delivery system 10, causing regulator 70 (FIG. 5) to release fluid from containment device 50 at a predetermined pressure to delivery system 10 downstream of regulator 70. Application device 20 may be, e.g., a garden-hose handle or other pistol-like configuration. Actuating devices 22, 24 may be any push button, trigger mechanism, or other device that, when actuated, opens a valve and releases fluid, as will be described in greater detail herein and in incorporated by reference U.S. application Ser. No. 16/589,633.

With continued reference to FIG. 1, cap 32 is configured to attach to a distal end of handle 30 such that cap 32 seals lumen 34 of handle 30 when cap 32 is attached to handle 30. Cap 32 may be attached to the proximal end of handle 30 using a threaded connection (e.g., threads 30b of cap 32 interacting with corresponding threads 30b of handle 30 in FIG. 4). For example, after containment device 50 is inserted into lumen 34, cap 32 may be placed at the proximal end of handle 30. An inner surface of a wall 32a of cap 32 (specifically, a bottom or proximalmost inner surface of wall 32a) may contact containment device 50. Cap 32 may be screwed onto handle 30 via the threaded connection, as discussed herein. As cap 32 is screwed onto handle 30, cap 32 may urge containment device 50 toward a pierce pin located at the inlet of application device 20 (e.g., inlet 76 in FIG. 5) and into fluid communication with application device 20. However, since a user cannot determine when containment device 50 is entirely in fluid communication with application device 20, it may be dangerous for a user to activate one or more triggers 22, 24 of application device 20 before application device 20 is fully energized or charged with propellant fluid. Thus, it may be necessary to provide one or more indicators to advise the user when fluid containment device 50 is in complete fluid attachment to application device 20 such that application device 20 is energized or charged with propellant fluid from containment device 50.

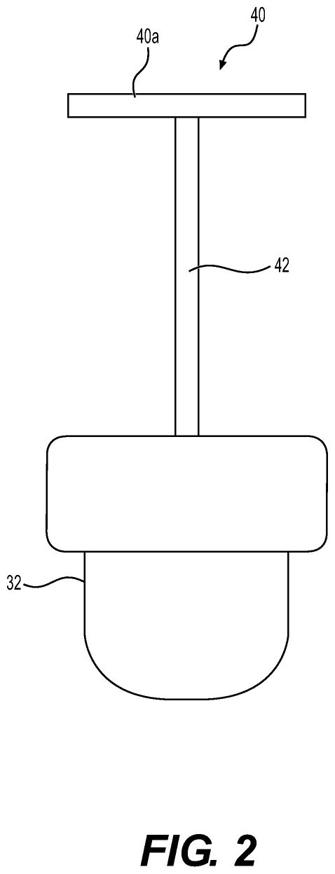

With reference to FIGS. 2 and 4, an indicator 40 according to an example is shown. Indicator 40 is connected to the inner surface of wall 32a of cap 32 via a post 42. Post 42 may be connected to and extend from cap 32 at a first end, and attached to indicator 40 at a second, opposite end. It will be understood that post 42 may extend further than indicator 40 in the distal direction. Indicator 40 may be a ring-shaped (annular-shaped) member having an opening therein to receive containment device 50. A diameter of the opening of the annulus of indicator 40 is greater than an outer diameter of wall 50a of containment device 50. An outer diameter of the annulus of indicator 40 is less than an inner diameter of a wall 30a of handle 30 (FIG. 4). While indicator 40 is shown as a complete ring-shaped member, unbroken in 360-degrees, it will be understood that indicator 40 may be C-shaped and therefore discontinuous about its circumference. For example, indicator 40 may be only a partial ring, having material extending 90-degrees, 180-degrees, 270-degrees, or some other amount around a circumference. In addition, or alternatively, a plurality of posts 42 may extend from cap 30, and each post 42 may be attached to corresponding indicators 40, or posts 42 may be attached to a single indicator 40 for, e.g., added support. It will be understood that posts 42 are off-centered so as not to interfere with containment device 50.

Indicator 40 may also include markings, colors, and/or other indications on a radially outer-facing surface thereof (a surface facing wall 30a). For example, indicator 40 may include the word ENERGIZED on an outer surface 42 of indicator 40. As will be explained herein, when a user is able to read the entire word ENERGIZED in window 36 of handle 30, the user may understand that application device 20 is ready for use. It will be understood that the term is not limited to ENERGIZED and any other words, terms, numbers, or symbols may be used, such as, e.g., READY, PRESSURIZED, or the like. Additionally, or alternatively, indicator 40 may include a green band (or a different colored band) on the outer surface of indicator 40 at a proximalmost end (e.g., the end closest to cap 32). When the green band is visible in the window of handle 30, it may be understood that application device 20 is ready for use. Alternatively, an outer surface of containment device 20 may have a color, e.g., red. As cap 32 is advanced onto handle 30, indicator 40 may block a user's view of containment device 50 through window 36 in handle 30. Once containment device 50 is no longer viewable in window 36, it may be understood that application device 20 is ready for use. It will be understood that indicator 40 may move relative to containment device 50 at a rate equal to a thread pitch of threads 30b and 32b.

Referring to FIGS. 3A-3C and 4, attachment of cap 32 to handle 30 is shown. Containment device 50 may be inserted through the distal end of handle 30 into lumen 34. Indicator 40 and a portion of post 42 are inserted into lumen 34, as shown in FIG. 4, such that indicator 40 is radially outward of sidewall 50a of containment device 50, and radially inward of sidewall 30a of handle 30. Indicator 40 may be advanced distally, e.g., in a direction indicated by arrow M in FIGS. 3A-3C, such that threads 32b of cap 32 contact threads 30b of handle 30 (FIG. 4). In a first position shown in FIG. 3A, indicator 40 is not viewable in window 36, such that the entirety of window 36 is shown by a non-energized indicator 36a (e.g., a view of an outer surface of sidewall 50a of containment device 50, which may have a color as described herein).

A user may twist cap 32 onto handle 30 via threads 32b of cap 32 and threads 30b of handle 30. As cap 32 is twisted in a direction indicated by arrow L in FIG. 3B, an inner, proximalmost surface of sidewall 32a may contact an outer, proximalmost surface of sidewall 50a and urge containment device 50 in the distal direction, toward the pierce pin. As cap 32 is twisted onto handle 30 in the direction indicated by arrow L, indicator 40 moves distally and may be partially viewed in window 36 (see FIG. 3B).

Continued twisting of cap 32 may cause containment device 50 to be urged into complete connection with the pierce pin. When cap 32 is completely twisted onto handle 30, containment device 50 may be in complete connection with the pierce pin, and application device 20 may be energized with propellant fluid. At this position, indicator 40 completely fills window 36, and non-energized indicator 36a is no longer viewable via window 36. In this position, the user may see a term, e.g., ENERGIZED, or a colored band, e.g., a green band, indicating that application device 20 is fully energized or pressurized and ready for use.

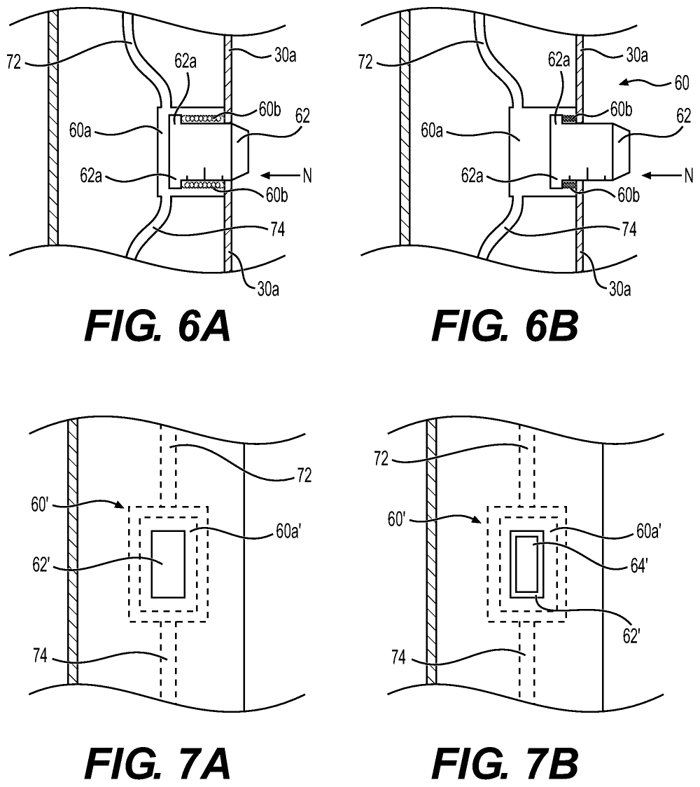

An indicator according to another example is shown in FIGS. 5, 6A, and 6B. Application device 20 may again include handle 30 and cap 32, and containment device 50 may be inserted into the lumen of handle 30. Alternatively, cap 32 may be a lever device for urging containment device toward the pierce pin arranged in or proximal to tube 74 of application device 20. The pierce pin may interact with opening 76 of containment device 50. Opening 76 may include a seal (e.g., a rubber seal, a resin seal, or any other seal suitable for maintaining a pressure of the propellant fluid within containment device 50). Once the pierce pin pierces the seal at opening 76, propellant fluid flows into tube 74 of application device 20. To determine when application device 20 is energized, indicator 60 may be arranged in fluid connection with the fluid path from containment device 50, through application device 20, and to the target site via tube 100. As shown in FIG. 5, indicator 60 may be upstream of regulator 70, and connected to regulator 70 via tube 72. In this manner, indicator 60 may determine when application device 20 is energized without any intervening structures, e.g., regulator 70, triggers 22, 24, or other intervening structures.

With reference to FIGS. 6A and 6B, indicator 60 includes a protrusion 62 which may extend from a sidewall of application device 20 (e.g., sidewall 30a of handle 30). Protrusion 62 may protrude from handle 30, e.g., a proximal end of handle 30 closer to actuators 22, 24, or may protrude from another surface of application device 20. Protrusion 62 may be any cross-sectional shape, e.g., spherical, rectangular, or the like. Indicator 60 includes a housing defining a cavity 60a. Protrusion 62 may move within cavity 60a from a position within application device 20, to a position extended radially outward from application device 20. Protrusion 62 may form a plunger or similar structure, and may taper from a radially inner surface toward a radially outward surface of protrusion 62. A radially inner end of protrusion 62 may include a flange 62a connected to sidewall 30a of handle 30 via one or more springs 60b. Outer surfaces of flange 62a (shown at the top and bottom in FIGS. 6A and 6B) may contact and seal against inner surfaces of cavity 60a that face those flange surfaces. In this manner, pressurized fluid will remain on one surface of flange 62a (the left side in FIGS. 6A and 6B) and not flow to, and press against, an opposite surface adjacent springs 60b Springs 60b may bias protrusion 62 into cavity 60a in a direction indicated by arrow N (e.g., a first position). Pressurized fluid may be supplied to cavity 60a via tube 74. The pressurized fluid may overcome the spring force of springs 60b, which may cause projection 62 to move in a radially outward direction, e.g., opposite the direction indicated by arrow N. When the pressurized fluid overcomes a threshold, projection 62 may be in a second position, e.g., shown in FIG. 6B, which may indicate that delivery system 10 is fully pressurized.

As shown in FIG. 6B, an outer surface of protrusion 62 may include markings 62b. Markings 62b may be a word or term, e.g., the word ENERGIZED as described herein. Alternatively, or additionally, markings 62b may include a green band on a radially innermost end of protrusion 62. As another example, markings 62b may be similar to empty/full fuel markings in a car, e.g., to indicate when a small amount of propellant fluid is passing into application device 20 or when application device 20 is energized. As containment device 50 is connected to application device 20, indicator 60 may move from a first position, shown in FIG. 6A, where protrusion 62 is completely, or partially, disposed within application device 20 to indicate that no propellant fluid is within application device 20, to a second position, shown in FIG. 6B, where protrusion 62 is completely exposed from application device 20, or otherwise exposed from device 20 more than in the first position, to indicate that application device 20 is energized. It will be understood that application device 20 may not be immediately energized, and propellant fluid may need to build up within application device 20, e.g., in the fluid path before regulator 70. In this situation, protrusion 62 may extend only partially from application device 20, or may be exposed an amount between its exposure in the first and second positions, until application device 20 is energized. For example, the fluid pressure in cavity 60a may be sufficient to overcome a fraction of the spring force exerted by springs 60b to move protrusion 62 only a portion of the distance to the second position in the radially outward direction. Projection 62 may extend completely from application device 20, or otherwise be in the second position, when the pressure of the propellant fluid is above a threshold.

It will also be understood that indicator 60 may indicate when containment device 50 (and, thus, application device 20) is low on propellant fluid. For example, indicator 60 may be a pressure gauge and the pressure of the fluid in cavity 60a may overcome the spring force of springs 60b and cause protrusion 62 to move radially outward in the direction opposite of arrow N when pressurized fluid passes through indicator 60. If the pressure of propellant fluid from containment device 50 decreases, e.g., such that the pressure of the fluid in cavity 60a is below the threshold, projection 62 may move radially inward in the direction of arrow N, such that only a portion of projection 62 may extend from application device 20. In this manner, a user may understand both when application device 20 is energized, and when an amount of propellant fluid passing into application device 20 is insufficient to deliver a medical agent from application device 20 to the body target.

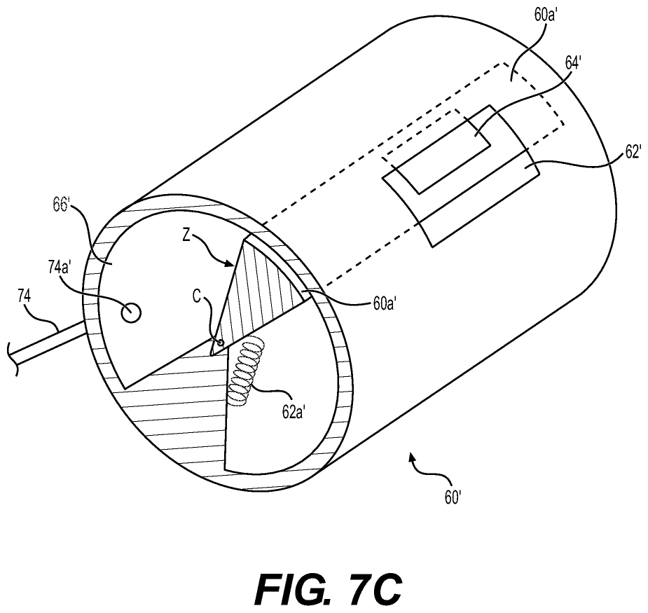

An example of another indicator 60′ is shown in FIGS. 7A-7C. Indicator 60′ may be positioned in handle 30 in a same position as indicator 60. Indicator 60′ may include a cylinder having a movable member 60a′ within a housing. FIG. 7C shows the cylinder with a bottom wall removed to show the inner components. The cylinder may be a portion of handle 30, so that the cylindrical wall is comprised of an outer wall of handle 30. Movable member 60a′ may rotate as a pressurized propellant fluid flows through indicator 60′. A window 62′ may be disposed in a sidewall of application device 20 which may allow a user to view movable member 60a′ at it rotates in the housing. For example, a portion of movable member 60a′ viewed through window 62′ may be the same color as handle 30 or may be a color, e.g., red, when containment device 50 is not attached to application device 20, as shown in FIG. 7A.

Once containment device 50 is attached to application device 20 and propellant fluid begins to flow into application device 20, movable member 60a′ may rotate about its axis C, shown in FIG. 7C, to a position shown in FIG. 7B, in which one or more markings 64′ on the sidewall of cylinder 60a′ are viewable within window 62′. For example, FIG. 7C illustrates indicator 60′. Indicator 60′ may be a cylinder having movable member 60a′ which rotates about axis C. Indicator 60′ may include a cavity 66′ which may receive the propellant fluid from tube 74 via an opening 74a′ in indicator 60′. Fluid may exit cavity 66′ via tube 72 (not shown in FIG. 7C) to travel downstream in the fluid path of delivery system 10. As the pressure of the fluid increases in cavity 66′, movable member 60a′ may rotate about axis C in the direction indicated by an arrow Z. One or more springs 62a′ (only one spring 62a′ shown in FIG. 7C) may bias movable member 60a′ in a position (e.g., a first position) opposite the direction indicated by arrow Z. When the pressure of the fluid is sufficient to overcome a spring force of spring 62a′, movable member 60a′ may be moved to a second position (in the direction indicated by arrow Z).

For example, FIG. 7B may illustrate an energized state of application device 20, where movable member 60a′ is in the second position. As described herein, markings 64′ may be any marking, color, word, or the like to indicate that application device 20 is energized with propellant fluid (e.g., the pressure of the propellant fluid is greater than an energization threshold). As with indicator 60, indicator 60′ may indicate when only some propellant fluid is flowing into application device 20, or when a pressure of the propellant fluid entering application device 20 is below a threshold. For example, if the pressure of propellant fluid drops below a threshold, movable member 60a′ may rotate only partially such that only a portion of marking 64′ may be viewable within window 62′. In this scenario, the fluid pressure in cavity 66′ is insufficient to completely overcome the spring force of springs 62a′. In this manner, a user may determine when propellant fluid is flowing through application device 20, and when application device 20 is energized. It will be understood that window 62′ may include a covering or the like, similar to window 36.

While delivery system 10 is described as having cap 32 for urging containment device 50 toward the pierce pin, it will be understood that other mechanisms may be used. For example, cap 32 may include a lever action, as opposed to a threaded connection. Containment device 50 may be urged toward the pierce pin by a lever moving cap 32 and/or urging a proximal end of containment device 50 toward the pierce pin. In this situation, the indicator may be attached to a base of the lever to move within lumen 34 of handle 30 as the lever is moved. Further, while described separately, it will be understood that each of indicators 40, 60, and 60′ may be used by itself, or may be used in combination with one or more of the indicators described herein. It will also be understood that, in some instances, actuators 22, 24 are actuatable only when delivery system 10 is energized. For example, one or more indicators may prevent actuation of actuators 22, 24 if delivery system 10 is not energized.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A device for delivering pressurized fluid, the device comprising:
a handle having a lumen configured to receive a container of pressurized fluid;
a cap configured to close the lumen; and
an indicator configured to move from a first position to a second position, wherein the first position indicates disconnection of the container from a fluid path, and the second position indicates connection of the container to the fluid path.

2. The device according to claim 1, further comprising the container, wherein the lumen is outside the fluid path.

3. The device according to claim 2, further comprising:
a pierce pin within the handle and configured to pierce a seal of the container.

4. The device according to claim 1, wherein the indicator includes one or more markings configured to indicate when the container is in the second position, wherein the one or more markings are visible to a user when the container is in the second position and not visible to the user when the container is in the first position.

5. The device according to claim 1, wherein the indicator is connected to the cap via an elongate member, and wherein the elongate member is radially offset from a longitudinal axis of the handle.

6. The device according to claim 1, wherein the indicator includes an annular member, and wherein the indicator is configured to move relative to the handle and the container as the indicator moves between the first position and the second position.

7. The device according to claim 6, wherein an opening in the annular member accommodates the container.

8. The device according to claim 1, wherein a diameter of the indicator is greater than an outer diameter of the container and less than an inner diameter of the handle

9. The device according to claim 1, wherein the indicator includes one or more springs configured to bias the indicator in the first position.

10. The device according to claim 9, wherein the pressurized fluid is configured to overcome a spring force of the one or more springs to move the indicator from the first position to the second position.

11. The device according to claim 1, wherein the indicator includes a movable member configured to rotate about an axis within a housing based on a pressure of the pressurized fluid flowing into the device from the container.

12. The device according to claim 1, wherein the indicator includes a projection configured to move in a direction radially outward of the handle when the pressurized fluid flows through the fluid path.

13. The device according to claim 12, wherein the indicator is configured to move from the second position to a third position, the third position being between the first position and the second position to operate the device.

14. The device according to claim 1, wherein the device further includes an actuator configured to release the fluid from a downstream end of the device, and wherein the actuator is configured to be actuatable only when the indicator is in the second position.

15. The device according to claim 1, wherein the indicator is configured to indicate the pressurized fluid is flowing from the container at a rate sufficient to operate the device when the indicator is in the second position.

16. A device for delivering pressurized fluid, the device comprising:
a handle;
a cap configured to close an opening in the handle; and
an indicator attached to the cap, wherein the indicator includes a ring-shaped member configured to move relative to the handle when the cap is attached to the handle, and wherein the indicator is configured to indicate connection of a fluid-containing container to a fluid path of the handle.

17. The device according to claim 16, wherein an opening in the ring-shaped member accommodates the container.

18. The device according to claim 16, wherein the cap and the indicator are each configured to rotate relative to the handle.

19. The device according to claim 16, wherein an elongate member extends from and is attached to the cap at a first end, wherein the elongate member is attached at a second end, opposite the first end, to the indicator, and wherein the elongate member is offset from a central longitudinal axis of the cap.

20. A method for delivering pressurized fluid to a device, the method comprising:
inserting a container of pressurized fluid into a handle of the device;
moving an indicator from a first position to a second position, wherein the first position indicates disconnection of the container from a fluid path of the device, and the second position indicates connection of the container to the fluid path of the device; and
actuating the device when the indicator is in the second position.
